(19) United States
(12) Patent Application Publication
(10) Pub. No.: US 2024/0041385 A1
(43) Pub. Date: Feb. 8, 2024

(54) MYOELECTRIC SENSOR ARRAY

(71) Applicant: SHINKO ELECTRIC INDUSTRIES CO., LTD., Nagano (JP)

(72) Inventor: Kazuhiro YOSHIDA, Nagano (JP)

(21) Appl. No.: 18/357,443

(22) Filed: Jul. 24, 2023

(30) Foreign Application Priority Data
Jul. 27, 2022 (JP) 2022-119907

(51) Int. Cl. A61B 5/389

(52) U.S. Cl. CPC A61B 5/389; A61B 2562/046; A61B 2562/222

(57) ABSTRACT

A myoelectric sensor array includes a plurality of myoelectric sensors, and a plurality of wiring members each electrically connecting corresponding two adjacent myoelectric sensors among the plurality of myoelectric sensors, wherein each of the plurality of the myoelectric sensors includes a substrate, a pair of myoelectric electrodes provided on the substrate, and a signal processing circuit electrically connected to the pair of myoelectric electrodes and at least one of the wiring members.

### CROSS-REFERENCE TO RELATED APPLICATIONS

The present application is based on and claims priority to Japanese Patent Application No. 2022-119907 filed on Jul. 27, 2022, with the Japanese Patent Office, the entire contents of which are incorporated herein by reference.

### FIELD

The disclosures herein relate to a myoelectric sensor array.

### BACKGROUND

Conventionally, myoelectric electrodes are attached to multiple parts of the living body to produce an electromyogram of a living body. Each myoelectric electrode is connected to a controller provided with a monitor or the like via an independent cable.

When the above-described conventional myoelectric electrodes are used, attaching the myoelectric electrodes to a living body is complicated.

Accordingly, there may be a need to provide a myoelectric sensor array that can reduce the complexity of attaching myoelectric electrodes.

### PRIOR ART DOCUMENT

[Patent Document]
[Patent Document 1] Japanese Laid-Open Patent Publication No. 2018-114093

### SUMMARY

According to at least one embodiment, a myoelectric sensor array is provided. The myoelectric sensor array includes a plurality of myoelectric sensors, and a plurality of wiring members each electrically connecting corresponding two adjacent myoelectric sensors among the plurality of myoelectric sensors, wherein each of the plurality of the myoelectric sensors includes a substrate, a pair of myoelectric electrodes provided on the substrate, and a signal processing circuit electrically connected to the pair of myoelectric electrodes and at least one of the wiring members.

### BRIEF DESCRIPTION OF DRAWINGS

FIGS. 1A and 1B are drawings illustrating a myoelectric sensor array according to a first embodiment;

FIG. 2 is a drawing illustrating a method of using the myoelectric sensor array according to the first embodiment;

FIG. 3 is a drawing illustrating a myoelectric sensor array according to a second embodiment; and

FIGS. 4A and 4B are drawings illustrating a method of using the myoelectric sensor array according to the second embodiment.

### DESCRIPTION OF EMBODIMENTS

Hereinafter, embodiments of the present disclosure will be described with reference to the accompanying drawings. In the specification and the drawings, the same components are denoted by the same reference numerals, and a duplicate description thereof may be omitted.

### First Embodiment

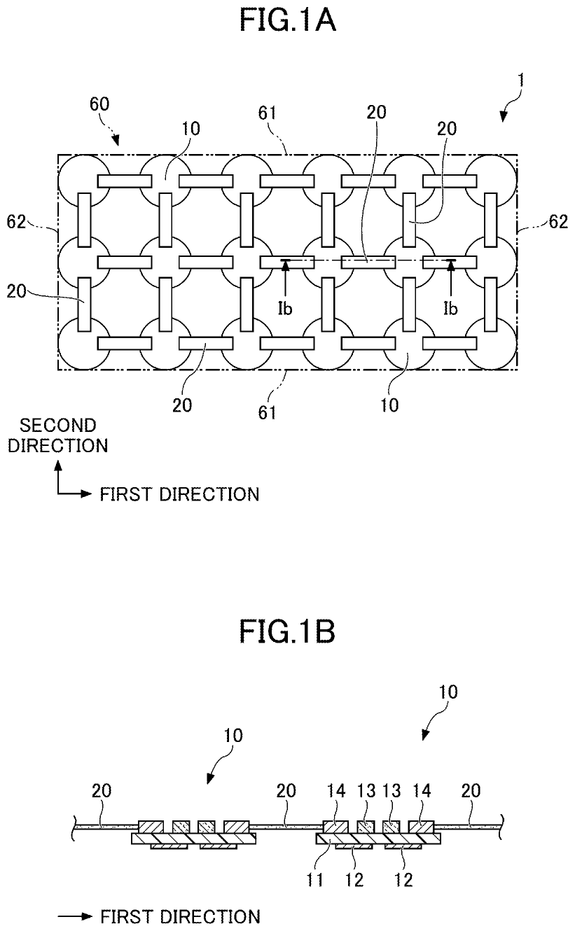

A first embodiment will be described. The first embodiment relates to a myoelectric sensor array. FIGS. 1A and 1B are diagrams illustrating a myoelectric sensor array according to the first embodiment. FIG. 1A is a plan view and FIG. 1B is a cross-sectional view. FIG. 1B corresponds to a cross-sectional view taken along a line Ib-Ib in FIG. 1A.

A myoelectric sensor array 1 according to the first embodiment includes a plurality of myoelectric sensors 10. The plurality of myoelectric sensors 10 is arranged in a first direction and a second direction perpendicular to the first direction. The second direction is a direction rotated counterclockwise by 90 degrees from the first direction. The myoelectric sensors 10 are arranged in a grid pattern, preferably in a square grid pattern. For example, a plurality of myoelectric sensors 10 in a first row arranged side by side in the first direction and a plurality of myoelectric sensors 10 in a second row arranged side by side in the first direction adjacent to the plurality of myoelectric sensors 10 in the first row are also arranged in columns in the second direction in plan view. The number of myoelectric sensors 10 arranged in the first direction is larger than the number of myoelectric sensors 10 arranged in the second direction. When assuming a rectangular region 60 that is the minimum region surrounding all of the myoelectric sensors 10, as viewed in a plan view direction that is perpendicular to the first direction and the second direction, two sides 61 of the rectangular region 60 are parallel to the first direction and perpendicular to the second direction, and the other two sides 62 are perpendicular to the first direction and parallel to the second direction. That is, in the present embodiment, the sides 61 and the sides 62 of the rectangular region 60 are not tilted from the first direction and the second direction.

The myoelectric sensor 10 includes a substrate 11, a pair of myoelectric electrodes 12, electronic components 13, and connectors 14. For example, the electronic components 13 and the connectors 14 are provided on one surface of the substrate 11, and the myoelectric electrodes 12 are provided on the other surface of the substrate 11. The number of electronic components 13 is not limited. The electronic components 13 constitute a signal processing circuit. The signal processing circuit includes, for example, a filter, a differential amplifier, and a switch. Each of the connectors 14 is electrically connected to at least one of the electronic components 13, and flexible printed circuit boards 20 described later are connected to the respective connectors 14. Each of the myoelectric electrodes 12 is electrically connected to at least one of the electronic components 13, and the signal processing circuit constituted by the electronic components 13 performs signal processing on an electrical signal such as a current flowing between the pair of myoelectric electrodes 12. The myoelectric sensor 10 may be referred to as an active electrode.

The myoelectric sensor array 1 includes a plurality of flexible printed circuit boards 20. Each flexible printed circuit board 20 electrically connects two myoelectric sensors 10 to each other. Specifically, some of the plurality of flexible printed circuit boards 20 extends in the first direction and electrically connects two adjacent myoelectric sensors 10 arranged side by side in the first direction. The rest of the flexible printed circuit boards 20 extends in the second direction and electrically connects two adjacent myoelectric sensors 10 arranged side by side in the second direction. Each flexible printed circuit board 20 includes, for example, a plurality of flexible insulating layers made of resin and an interconnect layer made of metal such as copper foil provided between the insulating layers.

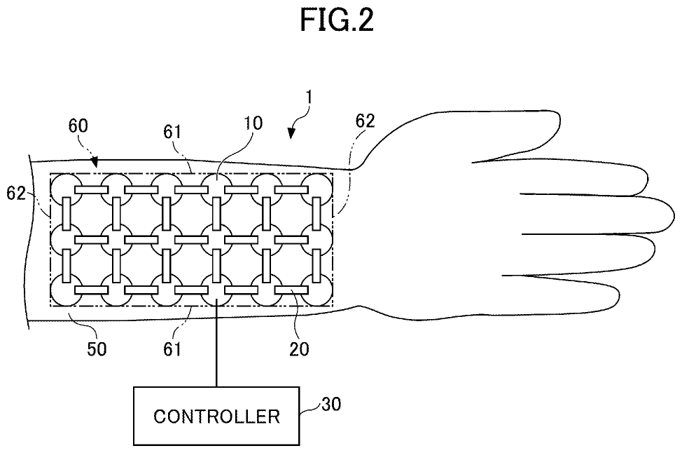

A method of using the myoelectric sensor array 1 will be described. FIG. 2 is a diagram illustrating a method of using the myoelectric sensor array 1 according to the first embodiment.

As shown in FIG. 2, the myoelectric sensor array 1 is attached to a living body in order that the myoelectric electrodes 12 of each myoelectric sensor 10 are in contact with the skin. For example, the myoelectric sensor array 1 is attached to an arm 50 of a human body. At this time, the sides 61 which are the long sides of the rectangular region 60 are set to be substantially parallel to the muscle fiber to be measured. The myoelectric sensor array 1 is connected to a controller 30 in a wired or wireless manner. In the case of wired connection, for example, one or two or more of the myoelectric sensors 10 located closest to the side 61 or the side 62 of the rectangular region 60 and the controller are connected by a cable or the like. In the case of wireless connection, for example, a wireless transceiver is mounted on any one of the myoelectric sensors 10, or a wireless transceiver is connected to the myoelectric sensor array 1.

Electric power is supplied to the myoelectric sensor array 1 from the controller 30 through the flexible printed circuit board 20. Under the control of the controller 30, any given myoelectric sensor 10 detects, via the myoelectric electrodes 12, an electrical signal generated in response to the movement of a muscle, and outputs the signal processed by the signal processing circuit to the controller 30 via one or more intervening flexible printed circuit boards 20 and one or more intervening myoelectric sensors 10 intervening between the given myoelectric sensor 10 and the controller 30. Processed electrical signals from the respective myoelectric sensors 10 are output to the controller 30 by sequentially scanning the myoelectric sensors 10 by, for example, the operation of the switches included in the signal processing circuits. In this way, the controller 30 can obtain the state of the muscle of the arm 50 using the myoelectric sensor array 1. The controller 30 can then produce, for example, an electromyogram.

According to the present embodiment, since the myoelectric sensor array 1 includes the plurality of myoelectric sensors 10 and two adjacent myoelectric sensors 10 among the plurality of myoelectric sensors 10 are connected to each other via one of the flexible printed circuit boards 20, attaching myoelectric electrodes to a living body is easy. Furthermore, the same number of cables between the myoelectric sensor array 1 and the controller 30 as the number of myoelectric sensors 10 are not required. The connection configuration between the myoelectric sensor array 1 and the controller 30 can therefore be simplified.

### Second Embodiment

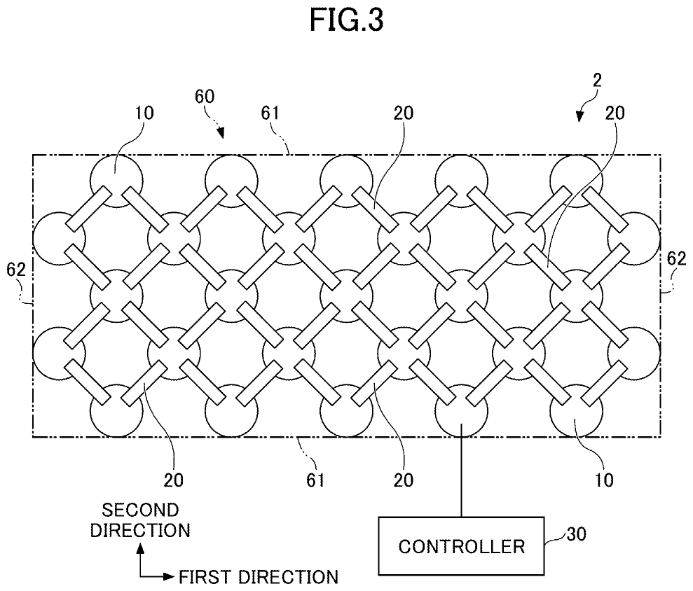

A second embodiment will be described. The second embodiment relates to a myoelectric sensor array. FIG. 3 is a plan view illustrating the myoelectric sensor array according to the second embodiment.

A myoelectric sensor array 2 according to the second embodiment includes, as in the first embodiment, a plurality of myoelectric sensors 10. The plurality of myoelectric sensors 10 is arranged in a first direction and a second direction perpendicular to the first direction. For example, a plurality of myoelectric sensors 10 in a first row arranged side by side in the first direction and a plurality of myoelectric sensors 10 in a second row arranged side by side in the first direction adjacent to the plurality of myoelectric sensors 10 in the first row are arranged in a zigzag pattern that alternates between the first row and the second row in the second direction in plan view. That is, the plurality of myoelectric sensors 10 is arranged in a staggered pattern in the first direction and the second direction. For example, the number of myoelectric sensors 10 arranged along the side 61 is larger than the number of myoelectric sensors 10 arranged along the side 62.

Similarly to the myoelectric sensor array 1, the myoelectric sensor array 2 includes a plurality of flexible printed circuit boards 20. Each flexible printed circuit board 20 electrically connects two myoelectric sensors 10 to each other. Specifically, each flexible printed circuit board 20 extends diagonally at 45 degrees with respect to the first direction and the second direction, and electrically connects two adjacent myoelectric sensors 10, among the plurality of myoelectric sensors, arranged side by side diagonally at a 45-degree angle.

Other configurations are the same as those of the first embodiment.

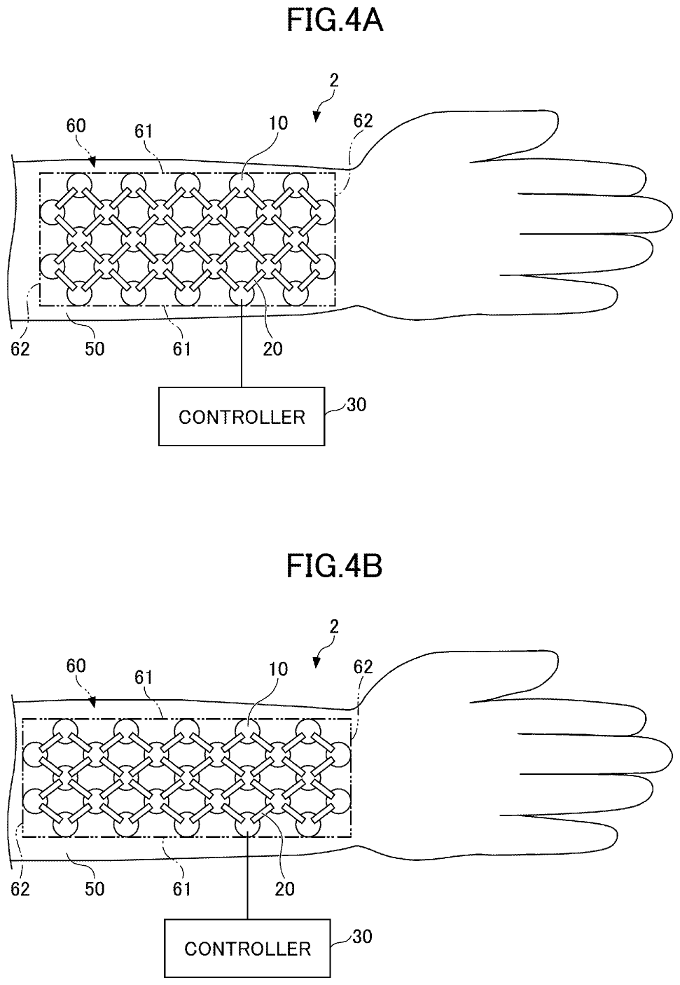

A method of using the myoelectric sensor array 2 will be described. FIGS. 4A and 4B are diagrams illustrating a method of using the myoelectric sensor array 2 according to the second embodiment.

As shown in FIG. 4A, similarly to the first embodiment, the myoelectric sensor array 2 is attached to an arm 50 of a human body in a manner such that sides 61, which are the long sides of a rectangular region 60, are substantially parallel to the muscle fiber to be measured and the myoelectric electrodes 12 of each myoelectric sensor 10 are in contact with the skin. The myoelectric sensor array 2 is connected to a controller 30 in a wired or wireless manner.

Similarly to the first embodiment, electric power is supplied to the myoelectric sensor array 2 from the controller 30 through the flexible printed circuit board 20. Under the control of the controller 30, any given myoelectric sensor 10 detects, via the myoelectric electrodes 12, an electrical signal generated in response to the movement of a muscle, and outputs the signal processed by the signal processing circuit to the controller 30 via one or more intervening flexible printed circuit boards 20 and one or more intervening myoelectric sensors 10 intervening between the given myoelectric sensor 10 and the controller 30.

According to the second embodiment, the same effect as that of the first embodiment can be obtained.

Further, even when a muscle moves during the measurement using the myoelectric sensor array 2, the myoelectric sensor array 2 is easy to deform in response to the movement of the muscle. For example, when a muscle stretches, the skin also stretches in a direction parallel to the muscle fiber. At this time, although stress acts on the myoelectric sensors 10 and the flexible printed circuit boards 20, the magnitude of the stress is small, and the myoelectric sensors 10 and the flexible printed circuit boards 20 are not substantially deformed. Furthermore, in the present embodiment, as shown in FIG. 4B, the myoelectric sensor array 2 is deformed so that the flexible printed circuit boards 20 become more parallel to the muscle fiber. The myoelectric sensor array 2 also expands in a direction parallel to the muscle fiber and contracts in a direction perpendicular to the direction. Thus, according to the second embodiment, excellent contractility can be obtained in the myoelectric sensor array 2.

In the second embodiment, the angle at which the flexible printed circuit boards 20 extend diagonally with respect to the first direction and the second direction is not limited to 45 degrees, but is preferably between 30 degrees and 60 degrees, inclusive, and more preferably between 40 degrees and 50 degrees, inclusive.

In both the first embodiment and the second embodiment, the long sides 61 of the rectangular region 60 are substantially parallel to the muscle fiber to be measured, but the short sides 62 of the rectangular region 60 may be substantially parallel to the muscle fiber to be measured. The myoelectric sensor array 1 or 2 may be attached so as to surround the entire circumference of the arm 50. Further, the myoelectric sensor array 1 or 2 may be formed in a cylindrical shape such as a supporter so as to surround the entire circumference of the arm 50. When the myoelectric sensor array 2 is formed in a cylindrical shape, the direction in which the myoelectric sensors 10 are arranged and the direction in which the flexible printed circuit board 20 expands are tilted relative to the axis of the array in the cylindrical shape.

As a wiring member, a cable may be used instead of the flexible printed circuit board 20.

According to the present disclosure, it is possible to reduce the complexity of attaching myoelectric electrodes.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment(s) of the present inventions have been described in detail, it should be understood that the various changes, substitutions, and alterations could be made hereto without departing from the spirit and scope of the invention.

What is claimed is:

1. A myoelectric sensor array, comprising:
a plurality of myoelectric sensors; and
a plurality of wiring members each electrically connecting corresponding two adjacent myoelectric sensors among the plurality of myoelectric sensors;
wherein each of the plurality of the myoelectric sensors includes:
a substrate;
a pair of myoelectric electrodes provided on the substrate; and
a signal processing circuit electrically connected to the pair of myoelectric electrodes and at least one of the wiring members.

2. The myoelectric sensor array as claimed in claim 1,
wherein the plurality of myoelectric sensors is arranged in a grid pattern in a first direction and a second direction perpendicular to the first direction,
wherein two adjacent myoelectric sensors, among the plurality of myoelectric sensors, arranged side by side in the first direction are connected to each other via one of the wiring members, and
wherein two adjacent myoelectric sensors, among the plurality of myoelectric sensors, arranged side by side in the second direction are connected to each other via one of the wiring members.

3. The myoelectric sensor array as claimed in claim 1,
wherein the plurality of myoelectric sensors is arranged in a staggered pattern in a first direction and a second direction perpendicular to the first direction, and
wherein the wiring members extend diagonally with respect to the first direction and the second direction, and electrically connect myoelectric sensors diagonally adjacent to each other.

4. The myoelectric sensor array as claimed in claim 3, wherein an angle at which the wiring members extend diagonally with respect to the first direction is between 30 degrees and 60 degrees, inclusive.

5. The myoelectric sensor array as claimed in claim 3, wherein an angle at which the wiring members extend diagonally with respect to the first direction is between 40 degrees and 50 degrees, inclusive.

6. The myoelectric sensor array as claimed in claim 1, wherein the wiring members are each a flexible printed circuit board or a cable.
